(19) United States
(12) Patent Application Publication
(10) Pub. No.: US 2014/0031538 A1
(43) Pub. Date: Jan. 30, 2014

(54) Systems, methods, and a kit for determining the presence of fluids

(71) Applicants: Justine S Chow, Durham, NC (US); James Garber Rudulph, JR., Durham, NC (US)

(72) Inventors: Justine S Chow, Durham, NC (US); James Garber Rudulph, JR., Durham, NC (US)

(21) Appl. No.: 14/042,052

(22) Filed: Sep. 30, 2013

Related U.S. Application Data
(63) Continuation-in-part of application No. 13/684,679, filed on Nov. 26, 2012.
(60) Provisional application No. 61/784,053, filed on Mar. 14, 2013.
(60) Provisional application No. 61/666,843, filed on Jun. 30, 2012.

(51) Int. Cl. C12N 15/10

(52) U.S. Cl. CPC C12N 15/1034
USPC 536/25.3

(57) ABSTRACT

This invention relates to nucleic-acid based product authentication and identification by determining authentication codes comprising target nucleic acids using oligonucleotide probes associated with samples. The presence of the authentication code is determined using detection methods, such as flow cytometric methods, capable of particle discrimination based on the light scattering or fluorescence properties of the particle. Target-correlated fluorescence signal, originating from a target nucleic acid hybridized to labeled complementary oligonucleotides is determined as an indicator of the presence of the authentication code. In some embodiments, an intercalating dye is used to determine the presence of target nucleotide/oligonucleotide heterodimers and identify an authentication code.

### CROSS REFERENCE TO RELATED APPLICATIONS

This application claims the benefit of U.S. Provisional Application No. 61/784,053, filed Mar. 14, 2013, and is a continuation-in-part of U.S. patent application Ser. No. 13/684,679, filed Nov. 26, 2012, which claims the benefit of U.S. Application No. 61/666,843, filed on Jun. 30, 2012, each of which is incorporated herein by reference in its entirety.

### BACKGROUND OF THE INVENTION

1. Field of the Invention

The present invention relates to methods for constructing a nucleic acid sequence for use in authenticating a sample, the method comprising randomly generating a plurality of nucleic acid sequences of a user-specified length such that the nucleic acid sequence has one or zero alignments with known biological and artificial sequences. The present invention further relates to determining the presence of fluids of interest, and more particularly, such determination being done through the use of DNA tracers.

2. Description of the Prior Art

Authenticating a sample to determine the source origination of the sample's constituents has proved to be a difficult and cumbersome task. Because of their intrinsic capability to carry diverse coded information, nucleic acids have been used to provide a secure, cost effective and forensic method to help companies track the flow of liquids through an environment.

Linking identification of specimen container contents to patient identification is a critical unmet need in clinical lab diagnostics. Because of their intrinsic capability to carry diverse coded information and their innate and nearly ubiquitous presence in biological specimens, nucleic acids have been used to provide a secure forensic method to help diagnosticians ensure that sample identification is correct. Typically, a biological specimen with an identity in question may be matched to an individual's identity if the DNA that occurs naturally in the specimen and the DNA from a sample known to come from the individual are sequenced and matched. This process offers identification at a high level of certainty, but typically at high costs in terms of expense, effort, and time.

Further, in relation to one embodiment of this invention, there is currently a lack of patented ideas that would embody DNA tracer technology stabile enough to withstand shearing forces to test groundwater without being toxic specifically aimed at hydraulic fracturing. Most hydraulic fracturing related patents and patent applications deal with oil recovery and the measure of backflow. No prior art is known to provide DNA tracers for detecting the presence of source specific fluids of interest in sampled liquids without contaminating the sampled liquids themselves, as with the present invention. Thus, there remains a need in the art to provide methods and systems for detecting the presence of fluids of interest in liquid samples in a safe and effective manner.

Microparticulate taggants have been used as means for authenticating products. For example, U.S. Pat. No. 7,874,489, filed Jun. 20, 2006 by Mercolino, discloses using such taggants, combining taggants that have different detectable physical properties, wherein each combination of properties is used as an encoding bit to create codes. Similarly to nucleic acids, unique taggants with unique combinations of physical properties must be manufactured in order to increase the number and complexity of possible codes.

The detection of nucleic acids is widely employed for determining the presence and copy number of specific genes and known sequences. An important characteristic of nucleic acids is their ability to form sequence-specific hydrogen bonds with a nucleic acid having a complementary nucleotide sequence. This ability of nucleic acids to hybridize to complementary strands of nucleic acids has been used to advantage in what are known as hybridization assays, and in DNA purification techniques. In a hybridization assay, a nucleic acid having a known sequence is used as a probe that hybridizes to a target nucleic acid having a complementary nucleic acid sequence. Labeling the probe allows detection of the hybrid and, correspondingly, the target nucleic acid.

Additional aspects of the invention include special design requirements for the nucleotide sequences in order to maximize their safety for use in products intended for intimate or parenteral contact with biological organisms, such as injectable pharmaceuticals, or ingested products, like certain pharmaceuticals, nutraceuticals, or foods.

### SUMMARY OF THE INVENTION

One embodiment of the invention is a method of constructing a nucleic acid sequence for use in authenticating a sample, the method comprising randomly generating a plurality of nucleic acid sequences of a user-specified length, comparing the plurality of nucleic acid sequences to known biological and artificial sequences, and selecting the nucleic acid sequences having one or zero alignments with the known biological and artificial sequences based on the comparison.

In another embodiment, the target nucleotide sequence or a plurality of target nucleic acid sequences that is or are intended for use in close association with biological organisms is designed so that they are dissimilar to known naturally occurring nucleotide sequences.

In another embodiment, methods and systems are disclosed for using a tracer for the detection of fluids of interest in suspected areas of contamination using biopolymers. Another embodiment of the present invention is to provide methods and systems for using a tracer for the detection of fluids of interest, wherein tracer variation permits origination sources to be distinguished from each other.

### BRIEF DESCRIPTION OF THE DRAWINGS

FIG. 1 shows a schematic of stop codon placement configuration options.

FIG. 2 is a flow chart containing the stepwise procedure for selecting nucleotide sequences that have minimized overlap with naturally occurring sequences and stop codons placed in one or more locations in the sequence.

FIG. 3 is a diagram of one embodiment of the invention, illustrating depictions of a secondary structure of the tracer using mfold software.

FIG. 4 is a diagram of the energy dot plot of the structure, shown in FIG. 3, illustrating the free energy of the tracer structure.

FIG. 5 is another software-generated diagram of one embodiment of the invention, illustrating DNA tracer structure.

### DETAILED DESCRIPTION

Sequence Generation: DNA sequences were designed for safe use as tracer ingredients in biological environments. Some examples of biological environments include biological specimens, products intended for injection like injectable pharmaceuticals, and substances that may enter a living ecosystem.

The first method of DNA sequence design involved the selection of sequences that have few or no occurrences in naturally occurring DNA. Because DNA encodes information that is used by living organisms for creating biological molecules, a DNA tracer sequence that has minimized similarity to the sequences found in nature is expected to be biologically inert by comparison, and therefore more safe than naturally occurring sequences.

The second method of DNA sequence design incorporated the additional requirement that one or more stop codons be incorporated with the DNA sequence. Stop codons terminate protein translation from DNA sequence, so their presence is expected to enhance biological inertness and therefore their safety. For demonstrative purposes, the sequences identified below include stop codons in a number of different locations relative to the sequence.

Sequence Set #1: DNA sequences designed for minimal similarity to naturally occurring DNA sequences. Using software written in the R language, a random number server (random.org) was used to generate random integers based on atmospheric noise. These numbers, integers between 1 and 4, were then translated to nucleic acid bases (“A,” “T,” “G,” “C”) and concatenated into sequences of a user-specified length (in this case, 35 nucleotides long). To ensure that sequences were unique and not present in nature, the sequences were batch-screened against the National Institute of Health BLAST database of known biological sequences, retaining four of those sequences with the fewest alignments.

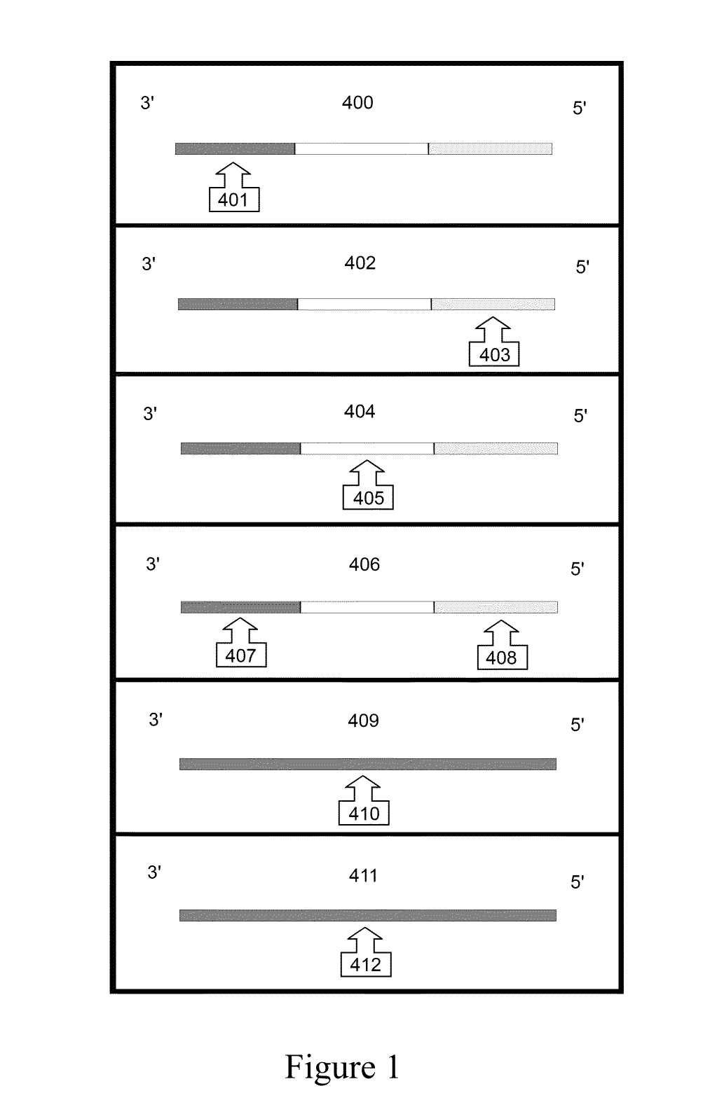

Sequence Set #2: DNA sequences designed for both minimal similarity to naturally occurring DNA sequences and having stop codons dispersed throughout. Using the R language, software was developed to generate random DNA sequence of user-specified length (in this case, 51 nucleotides long). One of three random DNA stop codons (“TAG,” “TAA,” or “TGA”) was then intentionally inserted into the sequences at specified points. The software was designed to insert stop codons using one of six ways (see FIG. 1), and a name was given to each of the categories:

First (400): A stop codon (401) is inserted within the first third of the sequence at a random point.

Third (402): A stop codon (403) is inserted within the latter third of the sequence at a random point.

Second (404): A stop codon (405) is inserted within the middle third of the sequence at a random point.

FirstThird (406): A stop codon is inserted within the first (407) and latter (408) thirds of the sequence at random points.

Complement (409): A stop codon complement (“ATC,” “ATT,” or “ACT”) is inserted within the first and latter thirds of the sequence at random points (410).

ThreeFrames (411): A random stop codon is inserted in each of the three reading frames (−1, 0, and +1) (412).

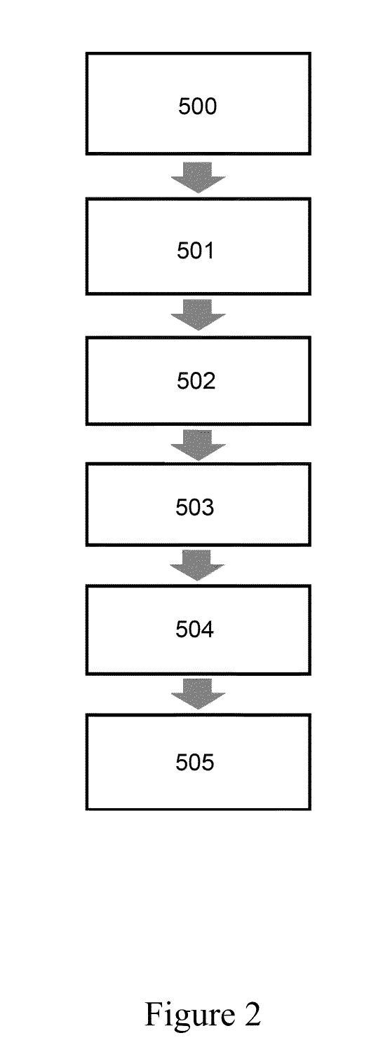

Sequences meeting both the non-similarity requirement and one of the stop codon requirements were screened for additional desirable properties, described in the following section and summarized by the flow chart shown in FIG. 2.

Sequence Screening. Step 1 (500—Random): 120 random sequences were generated over three runs using R software code. The sequences were then passed through four filters: NIH's BLAST alignment algorithm tool, a manual check for inserted and additional background stop codons, consecutive mononucleotides (base repeats), and a general analysis of secondary structure.

Step 2 (501—Insert Stop Codon): All random sequences generated by the R were then modified to include a stop codon in one of the six stop codon location configurations.

Step 3 (502—Non-Overlapping): Sequences from Step 2 were input into the National Institute of Health's Basic Local Alignment Search Tool (BLAST). Only those sequences having one or fewer alignments with known biological sequences were passed to Step 4.

Step 4 (503—Manual Stop Codon Search): For those sequences with only one alignment, the length of that alignment in terms of nucleotide base counts was determined. Next, we performed a manual search for target codons for each stop codon location category, and the number of total codons is counted. This count was recorded for later reference.

Step 5 (504—Consecutive Base Pair Search & Score): Sequences containing long nucleotide runs were screened. The number of runs for each of A, T, C, and G base pairs was counted. The longest run lengths were recorded for each base pair; the sum of run lengths for a single candidate sequence was used to create a preliminary unweighted score. Candidate sequences with lower scores were deemed to have a higher suitability for our purposes. Qualifying sequences were then passed to the next filter.

Step 6 (505—Secondary Structure Score): The candidate sequences were analyzed for secondary structure at standard conditions (37° C., 1M Na+). Sequences exhibiting fewer secondary structures (loops, pseudo knots) were considered more favorable, and the number of these structures was noted for each sequence. A final score was calculated for each candidate sequence by summing the total base pair score and secondary structures score. Representative sequences from each category having the lowest scores were chosen as approved sequences.

In one embodiment, one or more tracer oligonucleotides is or are associated with a fluid. The tracer oligonucleotides are associated with identifying characteristics for the fluid in a database. At a point after tracer addition to the fluid, the identity of the fluid can be determined by performing analysis using, by way of example and not limitation, a polymerase chain reaction (PCR) detection method.

In another embodiment, a method of constructing a nucleic acid sequence for use in authentication, is comprised of the following steps: (1) randomly generating a plurality of nucleic acid sequences of a user-specified length; (2) comparing the plurality of nucleic acid sequences to known biological sequences; and (3) selecting the nucleic acid sequences having fewer than 10 (ideally, one or zero) alignments with the known biological sequences based on the comparison. Further, this embodiment may include the additional step of incorporating at least one stop codon into each one of the plurality of nucleic acid sequences. Alternatively, an additional step may include incorporating at least one stop codon complement into each one of the plurality of nucleic acid sequences. Alternatively, additional steps may include determining the length of the alignment between the nucleic acid sequence and the known biological sequence, and excluding nucleic acid sequences having an alignment with a known biological sequence, wherein the alignment exceeds a predetermined length. Alternatively, additional steps may include determining a length of consecutive mononucleotide runs for each nucleotide in the nucleic acid sequence, and excluding nucleic acid sequences having at least one consecutive mononucleotide run exceeding a predetermined length. Alternatively, additional steps may include determining secondary structure for each of the plurality of nucleic acid sequences, and excluding nucleic acid sequences having a number of secondary structures exceeding a predetermined number. In the previous alternative, the secondary structure may, in one embodiment, be selected from the group consisting of loops and pseudo knots.

The present invention also allows for systems, methods, and a kit for use with hydraulic fracturing in relation to tracers and ground water or drinking water monitoring. While the prior art provides for improvements on DNA tracers used in testing and measuring liquids, none are known to be applicable to measure the safety level of drinking water whilst not contaminating the water. The present invention provides systems and methods and a kit for using a tracer for the detection of hydraulic fracturing fluid, including a method of creating well-specific tracers, and further a method of applying and interpreting water samples that may contain the tracer after the tracer's application to a hydraulic fracturing fluid. Several exemplar embodiments of the invention include using of the tracer for analyzing water rights, studying geological or environmental remediation, tracing industrial chemicals, waste or effluents, detecting the leakage of liquefied carbon dioxide, tracking the flow of liquids through a biological specimen, marking fuels in order to determine their origination, and tracing energy generation sources.

The tracer consists of nucleotide strands, which are biopolymers that consist of a sugar, phosphate group and nucelobases or nucleobase analogues. In preferred embodiments, nucleotides are used instead of nucleotide analogues. The nucleobase is a nitrogen-based molecule that, in DNA, forms hydrogen-bonded pairs that form the bridge between the two nucleotide strands. Nucleobase analogues include non-nitrogen bases that may not attach to each other, but are still able to form sequences along the length of the nucleotide strands. The nucleotide or nucleotide analogue also includes a five-carbon sugar, either ribose or 2-deoxyribose, and a phosphate group, PO43-. These resulting nucleotide strands contain sequences that can be customized as a unique tag for each individual tracer, designed for a specific well. The resulting strands are also able to form three dimensional (3-D) structures through specific hydrogen bonding formed from the sequences, increasing the compactness.

The 3-D structures, which can include hairpin structures, loops, or scaffolding configurations, decrease exposure to high shear and increases resistance to temperature or chemical degradation. Preferably, a single hairpin structure is provided that includes a base step loop section that confers durability and resilience, and a pair of dangling ends that are used for identification. The length of the strand and the distribution of specific types of nucleobases also increase the strand's strength. According to methods of the present invention, a tracer is mixed with water and added to a fluid of interest before they are injected into the origination source liquid or system. In other embodiments, the tracer may be mixed with other liquids more suitable for mixture with the fluids of interests. In one embodiment, material from a sample suspected to be contaminated by hydraulic fracturing fluids is later analyzed for the tracer. In such an embodiment, individual tracer sequences are matched to individual wells, identifying the exact well that is the source of contamination, thus providing well-specific tracers for identifying contamination by hydraulic fracturing fluids.

One embodiment of the present invention provides a method for determining the presence of fluids associated with a hydrocarbon reservoir used in hydraulic fracturing, including the steps of: synthesizing a tracer comprising a nucleotide or nucleotide analogue strand, wherein the tracer is capable of surviving hydraulic fracturing conditions; matching the sequence of the tracer with a specific well; diluting the tracer with water and inserting the mixture thus obtained into the hydraulic fracturing fluid; and analyzing environmental samples, such as groundwater, through methods such as polymerase chain reaction (PCR) or array-based electrical detection to determine whether the tracer is present.

Also, in such an embodiment, the tracer consists of nucleotide or nucleotide analogue sequences that inherently allow for variation diverse enough for each drilling well to have its own tracer within a drilling area. Preferably, the tracer may be diluted with water and added directly to the hydraulic fracturing fluid without needing any other additional materials more toxic than water. The tracer itself consists of material that is biologically inert and does not pose significant harm to biological systems; thus, the tracer is not more toxic to the environment than the hydraulic fracturing fluid to which it is being added. Also, the tracer is able to withstand the high salinity, acidic pH, and high metal ion content that is typically found in the surrounding fluid. Furthermore, the tracer is long enough and therefore durable enough to enable a detection temperature of above about 70° C. In another embodiment the tracer is long enough and durable enough to enable a detection temperature between about 70° C. and 100° C. Also, preferably, the tracer is able to form 3-D configurations that enable it to withstand shearing forces capable of pulling apart long unfolded sugar and phosphate chains.

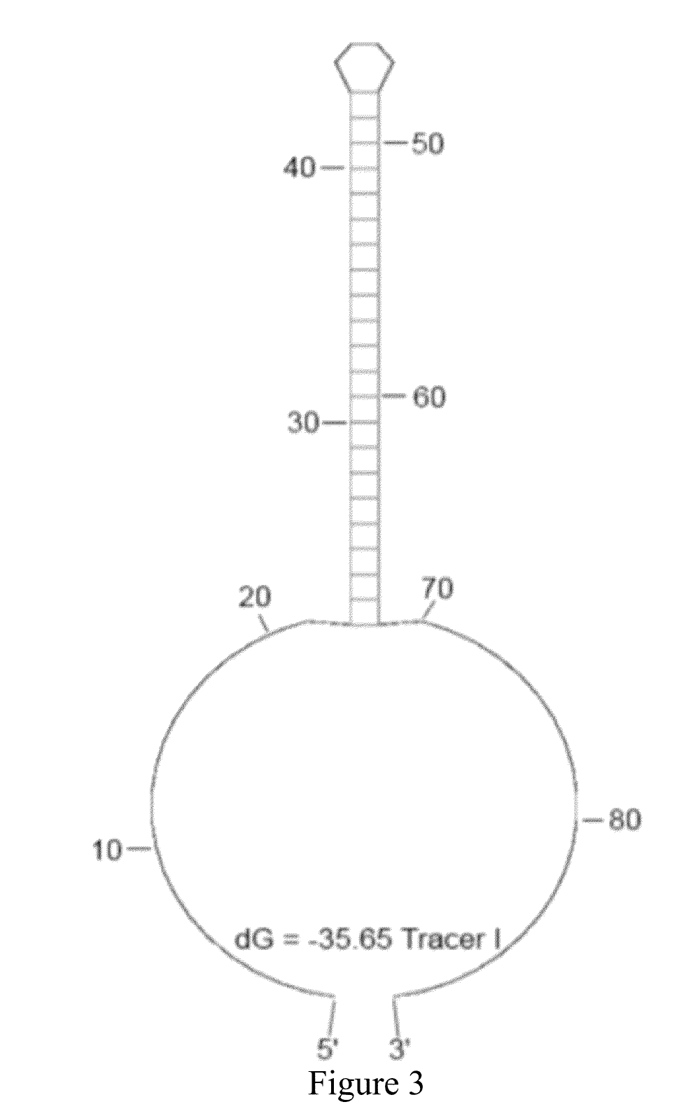

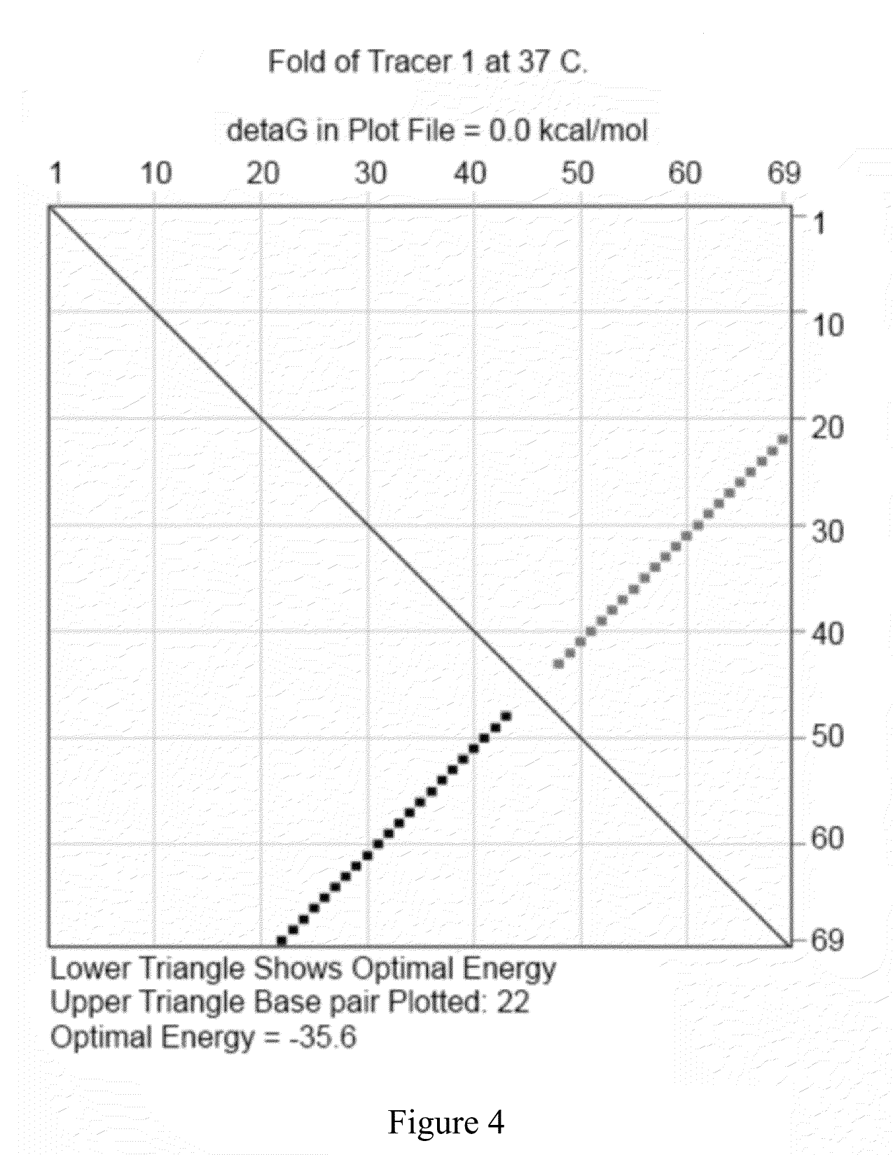

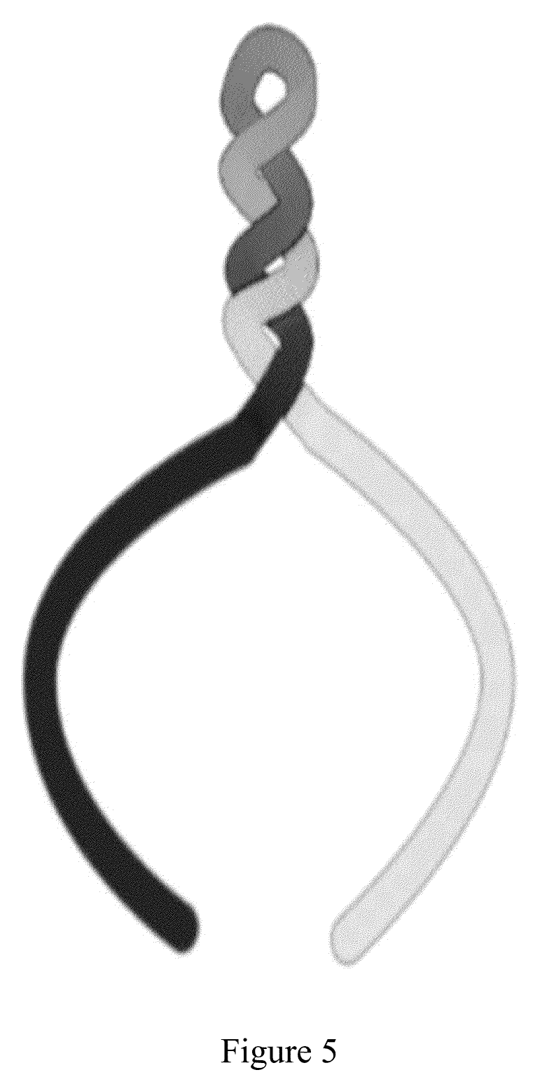

FIG. 3 is a diagram of one embodiment of the invention, illustrating depictions of a secondary structure of the tracer using mfold software. FIG. 4 is a diagram of the embodiment of the invention shown in FIG. 3, illustrating the free energy (AG) of the tracer structure. FIG. 5 is an illustrated three-dimensional diagram of one embodiment of the invention, showing the DNA tracer structure. In particular, as illustrated, the present invention uniquely provides DNA tracer methods, systems, and a kit used for detecting contamination of water or other fluid by hydraulic fracturing fluids, wherein the tracers include at least 60% G-C base pair content. Also, the tracer is characterized by an extremely strong “loop” at the middle of the sequence and a double-stranded stem that confers durability, while making the structure compact enough to withstand shearing forces. The tracer has unique identifier dangling ends that can be switched out for different wells for providing well-specific tracers. Notably, there is a hairpin structure of the tracer that unfolds at close to 100° C.; also, it does not degrade at higher temperatures.

In methods for molecule specification according to the present invention at least one DNA tracer is provided, wherein the tracer consists of a DNA sequence, a nucleic acid. Such a sequence is artificially synthesized and not found naturally according to National Institute of Health Basic Local Alignment Search Tool (BLAST). The DNA tracer is a single strand folded approximately in half, such that part of it is double stranded with another part of the strand, with a loop at its fold, forming a “hairpin” structure, and dangling ends that do not pair with the other ends and are free-floating single-stranded DNA, as illustrated in the FIGS. 1A, 1B, 1C.

In one embodiment of the invention, the method steps further include adding the tracer to hydraulic fracturing fluid during the regular mixing process for hydraulic fracturing. If there is a continuous stream from mixing to injection the tracer is mixed into the fluid near the beginning of injection. Flowback or produced water is provided for sampling and for confirmation that the tracer is present in the water.

In methods of using the tracers, systems and kits for testing groundwater according to the present invention, water samples are provided. These water samples are cleaned with an ethanol rinse for PCR inhibitor removal, sequences are amplified by polymerase chain reaction (PCR), and results are detected using a detection method, e.g., gel electrophoresis. Two sets of testing are provided: a first set of testing to detect the presence or absence of the DNA tracer(s) according to the present invention, as described hereinabove, either through a mix of multiple primers in PCR or through an universal tracer that interacts with the DNA tracer(s); and a second set of testing that is performed only in the case of a positive result or an uncertain result from the first set of testing. The second set of testing identifies which set of dangling ends were used with the DNA tracer(s) detected. The step of identifying the set of dangling ends includes isolating testing of individual pairs of primers in PCR and narrowing down or reducing the results to match a specific well (i.e., detecting well-specific tracers).

Certain modifications and improvements will occur to those skilled in the art upon a reading of the foregoing description. By way of example and not limitation, the methods, systems, and kit according to the present invention, while described for application to detection of contamination by hydraulic fracturing fluids, may be applied for detection and tracking of water rights, tracing groundwater or surface water systems for scientific analysis and tracing, for example the study of geology for environmental remediation, tracing chemicals, waste or other fluids for the purposes of accountability in other fields, carbon sequestration and/or detecting leakage of liquefied carbon dioxide, and/or tracing fuels. The above-mentioned examples are provided to serve the purpose of clarifying the aspects of the invention and it will be apparent to one skilled in the art that they do not serve to limit the scope of the invention. All modifications and improvements have been deleted herein for the sake of conciseness and readability but are properly within the scope of the present invention.

The invention claimed is:

1. A method of constructing a nucleic acid sequence for use in authenticating a sample, the method comprising:
randomly generating a plurality of nucleic acid sequences of a user-specified length;
comparing the plurality of nucleic acid sequences to known biological sequences; and
selecting the nucleic acid sequences having fewer than 10 alignments with the known biological sequences based on the comparison.

2. The method of claim 1, further comprising incorporating at least one stop codon into each one of the plurality of nucleic acid sequences.

3. The method of claim 1, further comprising incorporating at least one stop codon complement into each one of the plurality of nucleic acid sequences.

4. The method of claim 1, further comprising:
determining the length of the alignment between the nucleic acid sequence and the known biological sequence; and
excluding nucleic acid sequences exceeding a predetermined number of alignments with a known biological sequence, wherein the alignment exceeds a predetermined length.

5. The method of claim 1, further comprising:
determining a length of consecutive mononucleotide runs for each nucleotide in the nucleic acid sequence; and
excluding nucleic acid sequences having at least one consecutive mononucleotide run exceeding a predetermined length.

6. The method of claim 1, further comprising:
determining secondary structure for each of the plurality of nucleic acid sequences; and
excluding nucleic acid sequences having a number of secondary structures exceeding a predetermined number.

7. The method of claim 6, wherein the secondary structure is selected from the group consisting of loops and pseudo knots.
